(19) United States
(12) Patent Application Publication
(10) Pub. No.: US 2009/0149719 A1
(43) Pub. Date: Jun. 11, 2009

(54) System And Method For Performing Remote Patient Risk Assessment Through A Visual Analog Scale

(75) Inventors: Ramesh Wariar, Blaine, MN (US); Yunlong Zhang, Mounds View, MN (US); Haresh G. Sachanandani, Shoreview, MN (US)

Correspondence Address: PAULY, DEVRIES SMITH & DEFFNER, L.L.C., PLAZA VII- SUITE 3000, 45 SOUTH SEVENTH STREET, MINNEAPOLIS, MN, 55402-1630, US

(73) Assignee: CARDIAC PACEMAKERS, INC., St. Paul, MN (US)

(21) Appl. No.: 12/257,296

(22) Filed: Oct. 23, 2008

Related U.S. Application Data
(60) Provisional application No. 60/992,613, filed on Dec. 5, 2007.

(51) Int. Cl. A61B 5/00

(52) U.S. Cl. 600/300

(57) ABSTRACT

A system and method for integrating qualitative assessment into remote patient management through a visual analog scale is provided. A query is associated to a physiological condition. A visual analog scale includes a linear gradient and, at each end, descriptors for a range of subjective and continuous responses to the query. Assessment data for a patient is obtained. A medical device of the patient is interrogated and stored data is received. The query is displayed with the visual analog scale. An answer to the query includes a point selected between the ends of and along the linear gradient. A distance of the point from one end of the linear gradient is determined. The distance is quantified as a fixed value in proportion to the distance. A risk to the patient is assessed. The stored data and the fixed value are analyzed against the physiological condition to represent patient wellness.

### CROSS-REFERENCE TO RELATED APPLICATION

This non-provisional patent application claims priority under 35 U.S.C. §119(e) to U.S. Provisional Patent application, Ser. No. 60/992,613, filed Dec. 5, 2007, the disclosure of which is incorporated by reference.

### FIELD

The present invention relates in general to remote patient management and, specifically, to a system and method for performing remote patient risk assessment through a visual analog scale.

### BACKGROUND

A visual analog scale (VAS) subjectively measures a health characteristic across a continuum of values. For instance, a simple two-dimensional VAS presents a question with a gradient or line having numbers or word descriptors on opposite ends, such as “no pain” and “severe pain.” A patient picks a point along the scale reflective of the characteristic measured.

In comparison to questionnaires, VASs are time-efficient and self-integrating. Although helpful, questionnaires are typically time consuming and may not be indicative of overall patient-perceived well-being, as questions can be misunderstood or tangential. A properly designed VAS is not suggestive of an answer and can shed light on a patient's health, particularly where the patient is otherwise unwilling or unable to elaborate on a condition or disorder in words.

Although VAS scores are subjective, VAS measurements have empirically demonstrated a credible degree of association with foretelling impending heart failure events in a manner similar to objective bioimpedance measurements. M. Packer et al., Utility of Impedance Cardiography for the Identification of Short-Term Risk of Clinical Decompensation in Stable Patients with Chronic Heart Failure, JACC, Vol. 47, No. 11, pp. 2245-52 (2006), the disclosure of which is incorporated by reference. VAS scores thus provide a useful adjunct to patient care, although with practical limitations.

In isolation, a single VAS measurement may not be reflective of or sensitive to an improvement in one symptom cancelled by the worsening of another symptom. As well, VAS measurements may fluctuate from visit-to-visit and from caregiver-to-caregiver. An individual caregiver's style, approach, and even understanding may alter VAS results.

Soliciting VAS data more frequently, informally, and efficiently, such as at home using a monitoring device, can improve consistency. Caregivers have increasingly gained access to remotely measured physiometry through at-home monitoring devices that can help manage a chronic condition or a disease, such as heart failure. For example, patient-operable interrogators, commonly known as “repeaters” or “communicators,” enable caregivers to remotely gather hemodynamic data and general patient physiometry. This data can be supplemented with interactive questioning or VAS inquiries regarding a patient's perceived health.

Existing remote interrogators rely on questionnaires to obtain subjective patient information. For instance, U.S. Pat. No. 6,168,563, to Brown, discloses a system and method that enables a healthcare provider to remotely monitor and manage a health condition. Physiological monitoring devices, such as a blood glucose monitor or peak-flow meter, can be interfaced to supply patient data, which healthcare professionals can analyze, print, and display. Although patient queries can address specific healthcare concerns, Brown fails to gather information for subjectively perceived well-being by non-questionnaire means.

Thus, there is a need for an approach to remotely monitor and manage patient condition with reliable inquiry and collection of subjective self-assessments of perceived well-being.

### SUMMARY

One embodiment provides a system and method for performing remote patient risk assessment through a visual analog scale. A visual analog scale is defined and includes a gradient and descriptors for a continuous range of responses to a query. A user interface for a remotely-managed patient is provided. The query and the visual analog scale are provided to the patient. An answer to the query is accepted and includes a point subjectively selected by the patient along the gradient. The answer is quantified into a discrete value proportionate to a position of the point along the gradient determined from one of the ends. A risk to the patient is assessed and includes one of a status quo and change in condition by evaluating the discrete value against qualitative wellness criteria

A further embodiment provides a system and method for integrating qualitative assessment into remote patient management through a visual analog scale. A query is associated to an indication of at least one physiological condition. A visual analog scale is formed and includes a linear gradient and, at each end, descriptors for a range of subjective and continuous responses to a query. Assessment data for a remotely-managed patient is obtained. A medical device of the patient is periodically interrogated and stored data recorded by the medical device is received on a continuous basis. An interactive user interface for the patient is provided. The query is displayed with the visual analog scale. An answer to the query is accepted and includes a point selected by the patient between the ends of and along the linear gradient. A distance of the point from one end of the linear gradient is determined. The distance is quantified as a fixed value in proportion to the distance. A risk to the patient is assessed and includes one of a status quo and change in condition. The stored data and the fixed value are analyzed against the at least one physiological condition to represent patient wellness.

Still other embodiments will become readily apparent to those skilled in the art from the following detailed description, wherein are described embodiments of the invention by way of illustrating the best mode contemplated for carrying out the invention. As will be realized, the invention is capable of other and different embodiments and its several details are capable of modifications in various obvious respects, all without departing from the spirit and the scope of the present invention. Accordingly, the drawings and detailed description are to be regarded as illustrative in nature and not as restrictive.

### BRIEF DESCRIPTION OF THE DRAWINGS

FIG. 1 is a functional block diagram showing a system for performing remote patient risk assessment through a visual analog scale, in accordance with one embodiment.

FIG. 2 is a block diagram showing a patient-operable communicator.

FIG. 3 is a diagram showing, by way of example, a visual analog scale for remote patient self-assessment of dyspnea.

FIG. 4 is a process flow diagram showing a method for performing remote patient risk assessment through a visual analog scale, in accordance with one embodiment.

FIG. 5 is a flow diagram showing a visual analog scale processing routine for use in the method of FIG. 4.

FIG. 6 is a data flow diagram showing event risk evaluation for use with the routine of FIG. 5.

FIG. 7 is a data flow diagram showing post processing of a remote patient self-assessment for use with method of FIG. 4.

### DETAILED DESCRIPTION

### System

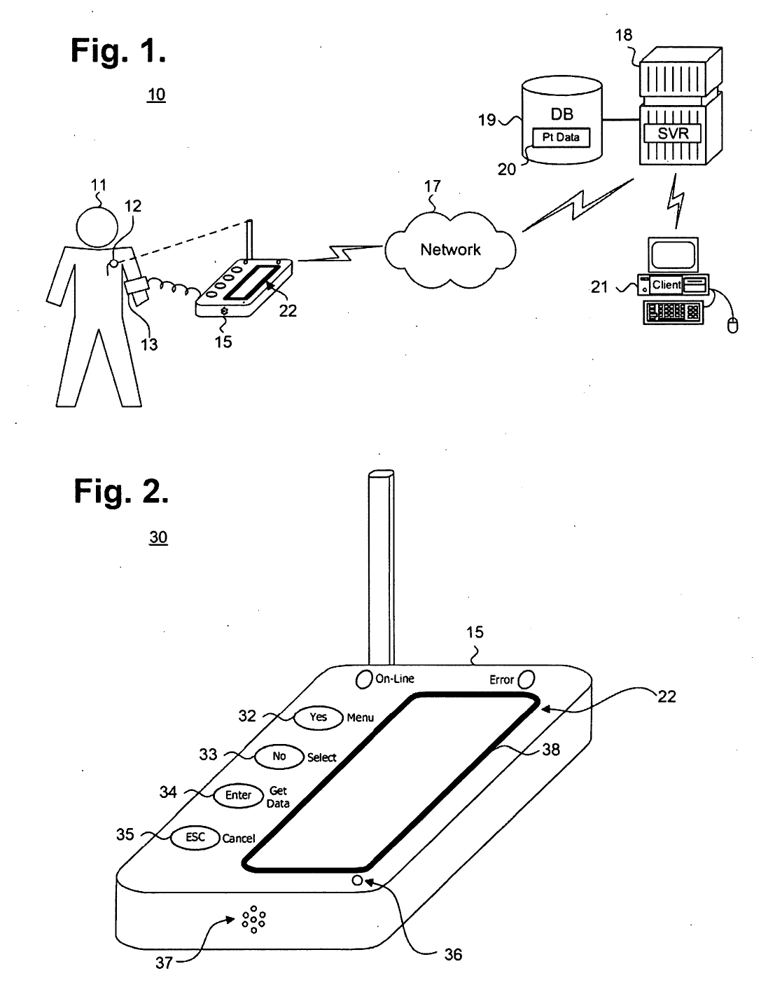

Self-assessments by patients are useful adjuncts to remote patient care. In particular, visual analog scales (VASs) provide an easily understood and user-friendly data collection approach that is amenable to unsupervised patient operation. FIG. 1 is a functional block diagram showing a system 10 for performing remote patient risk assessment through a VAS, in accordance with one embodiment. Remote patient care encompasses a wide range of services, including monitoring patient wellness, identifying significant changes in condition, recommending modifications to treatment regimen and medications, and alerting caregivers to areas of concern. Advanced remote patient care further includes determining whether a prescribed therapy satisfactorily addresses a managed condition, such as heart failure, diagnosing illnesses and health disorders, directly prescribing medications, and adjusting medical device operational parameters. Other types of services are possible.

The system 10 includes a patient-operable communicator 15 remotely interfaced to a centralized healthcare server 18 or other clinician-accessible facility over telephone line or an internetwork 17, such as the Internet. The communicator 15 can monitor the physiometry of a patient 11 and provides an interactive self-assessment as further described below beginning with reference to FIG. 2 through a user interface 22 that displays a VAS. The internetwork 17 is based on the Transmission Control Protocol/Internet Protocol (TCP/IP) protocol suite, although other protocol suites are possible. Additionally, other network topologies and configurations are possible.

Patient physiometry is obtained through external sensors 13. The external sensors can include sensors that remain in contact with the patient's body, such as a Holter monitor, as well as a wide range of medical and non-medical devices that the patient can use, operate, or upon which he can perform testing, such as a blood pressure cuff, weight scale, spirometer, skin resistance sensor, and the like. Internal sensors (not shown) can similarly be provided integral or connected to an IMD 12. Other types of sensors are possible. The sensors can be integral with or connected to the communicator 15 by wired or wireless means, such as inductive telemetry, radio frequency (RF) telemetry, or other forms of wireless telemetry based on, for example, “strong” Bluetooth or IEEE 802.11 interfacing standards. Other types of connection interfaces are possible.

Patient physiometry can also be obtained through implantable medical devices (IMDs) 12 that are permanently or temporarily introduced into a patient's body. Such devices include IMDs that are fully introduced into a patient's body, which include therapy delivery devices, such as pacemakers, implantable cardioverter-defibrillators, biventricular pacemakers, drug pumps, and neuro-stimulators; and physiometric monitoring devices, such as cardio or pulmonary sensors and monitors. Such devices also include IMDs that are partially introduced into a patient's body, which include physiometric monitoring devices, such as electroencephalogram recorders consisting of an extracorporeal recording device and electrodes that are placed subdurally or in the cerebral cortex. Other types of IMDs are possible.

Collected physiometry and qualitative self-assessment data are stored by the server 18 into a database 19 as patient data 20. The server 18 is a server-grade computing platform configured as a uni-, multi- or distributed processing system, which includes those components conventionally found in computing devices, such as, for example, a central processing unit (CPU), memory, network interface, persistent storage, and various components for interconnecting such components. Healthcare providers access the patient data 20 and other information through client devices 21, such as personal computers.

In a further embodiment, the patient data can be evaluated, either by an IMD 13, communicator 15, server 18, or other processing device for the occurrence of one or more chronic or acute health conditions, such as described in related, commonly-owned U.S. Pat. No. 6,336,903, to Bardy, issued Jan. 8, 2002; U.S. Pat. No. 6,368,284, to Bardy, issued Apr. 9, 2002; U.S. Pat. No. 6,398,728, to Bardy, issued Jun. 4, 2002; U.S. Pat. No. 6,411,840, to Bardy, issued Jun. 25, 2002; and U.S. Pat. No. 6,440,066, to Bardy, issued Aug. 27, 2002, the disclosures of which are incorporated by reference.

In a further embodiment, the patient data is extracorporeally safeguarded against unauthorized disclosure to third parties, including during collection, assembly, evaluation, transmission, and storage, to protect patient privacy and comply with recently enacted medical information privacy laws, such as the Health Insurance Portability and Accountability Act (HIPAA) and the European Privacy Directive. At a minimum, patient health information that identifies a particular individual with health- and medical-related information is treated as protectable, although other types of sensitive information in addition to or in lieu of specific patient health information could also be protectable.

### Communicator

Qualitative patient information is obtained through patient interaction using a communicator 15. FIG. 2 is a block diagram showing a patient-operable communicator 15 for use with the system 10 of FIG. 1. The communicator 15 is configured for patient or assisted operation in an at-home or clinical setting. The communicator 15 automatically reports patient data, including self-assessment results, to a centralized repository, such as a server 18 (shown in FIG. 1) or other caregiver-accessible facility via a telephone line, including land line and cellular, or an internetwork, such as described in commonly-assigned U.S. Pat. No. 7,009,511, issued Mar. 7, 2006 to Mazar et al., the disclosure of which is incorporated by reference. Other types of patient-operable devices with comparable physiometric and qualitative data collection and user interfacing capabilities could also be used

In general, communicators interrogate patients' medical devices, particularly IMDs, through wireless telemetry. Thus, the communicator 15 primarily functions as a medical device interrogation interface. During each interrogation session, the communicator 15 collects medical device-stored physiometry and other patient or device information for evaluation, relay, and storage. Additionally, the data can be post-processed to identify trends and for caregiver review, as further described below with reference to FIG. 7. Interrogation sessions preferably occur on a regular basis or as required.

The communicator 15 can also function as a collector of patient self-assessment information, either in combination with medical device interrogation or exclusively as a dedicated task. The communicator 15 includes an interactive user interface 22 with user input controls and output capabilities. The input controls include buttons 32-35, including a keypad; a touch-sensitive screen (not shown); a mouse, trackball, or other navigation and selection device (not shown); a microphone 36; or by other user manipulable device. Output capabilities include visual, tactile, or auditory outputs, such as a user display 38, vibration generator (not shown), and speaker 37, respectively. Other types of input controls and output capabilities are possible.

Patient self-assessment information is gathered via the user interface 22. The patient responds to conventional questionnaires regarding well-being and compliance. The questionnaires are supplemented with or, as appropriate, replaced by VASs presented on the display 31. VASs can be used for a variety of self-assessments, including heart failure (HF) status and diabetic conditions. Generally, a VAS is presented as a continuum along a horizontal or vertical line with numbers or word descriptors at each end. For instance, a question “How do you feel today?” would accompany a VAS labeled with “feeling good” and “feeling unusually fatigued and weak.” The patient picks a point along the VAS as a response, which is recorded as a self assessment.

VASs are particularly suited to self-assessments of secondary conditions or symptoms, for example, quality of life, dyspnea, blurry vision, drowsiness, shaking, trembling, sweating, heart palpitations, headache, dizziness, slurred speech, seizures, loss of consciousness, activities of daily life, exercise or activity impairment, and other types of pain and discomfort. Other VAS formats are possible, including two- or three-dimensional scales. Additionally, VASs offer several advantages over standard question-and-answer patient interchanges. VASs allow a patient to express a range of subjective inputs as one simple measure along a continuum. In contrast to questionnaires, a properly-designed VAS is relatively void of suggesting answers. When consistently analyzed in light of other objective and subjective measures, particularly for chronic conditions, VAS data can help to reliably predict the risk of an impending event, such as heart failure decompensation. The simplicity of VASs can also help monitor and urge patient compliance. Moreover, VASs can be extended to patients whose cognitive abilities are impaired or who cannot read by using images or symbols in place of words.

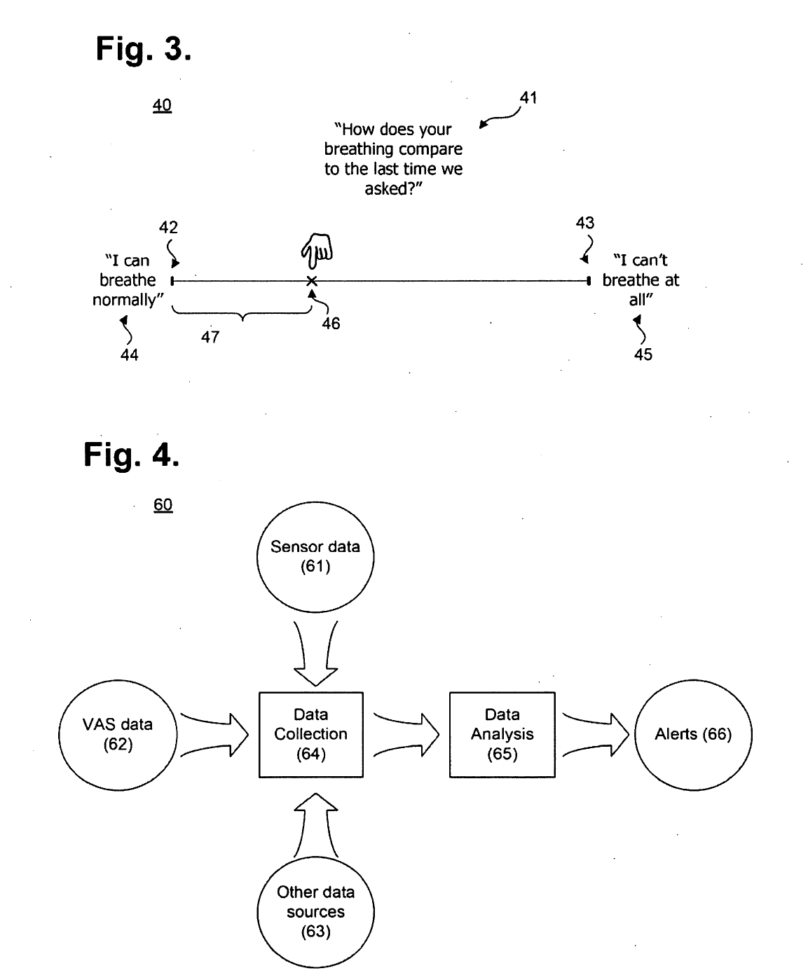

A VAS is presented to a patient as a visual query tool that is used in place of written answers or enumerated choices. In general, a VAS is displayed as a linear gradient, line, or scale. The endpoints of the VAS are labeled with numbers or descriptors. The patient answers an accompanying query by selecting a point 46 along the VAS, which indicates a range of subjective and continuous responses. FIG. 3 is a diagram showing, by way of example, a VAS 40 for remote patient self-assessment of dyspnea. Using the VAS 40, the patient 11 is asked a question 41 that he must answer by picking a point 46 along the VAS 40. The endpoints 42, 43 of the VAS 40 include word descriptors 44, 45 that define the range or continuum of possible answers.

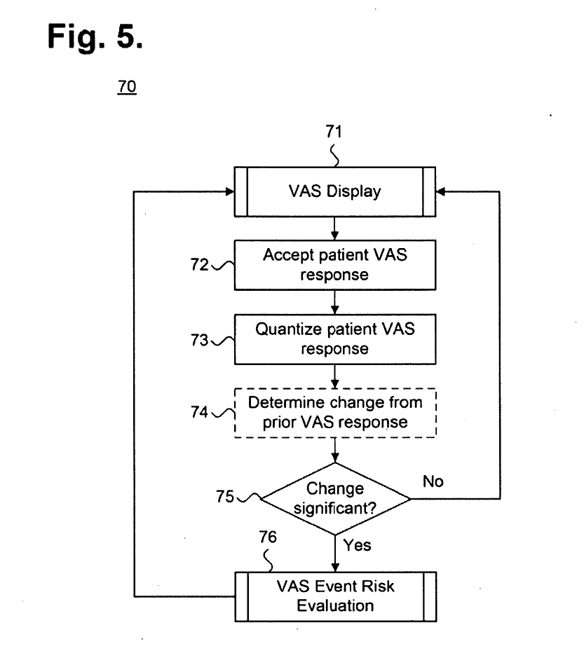

Each patient answer entered using the VAS 40 must be objectified into a quantitative value. A VAS 40 can be displayed as an uncalibrated and continuous range or with a scale numbered or labeled proportionate to the overall VAS. For instance, a VAS ranging from one to ten may have each even number labeled. To objectify or “quantize” each VAS response, the point selected by the patient as his answer is internally quantized into a numeric scale, typically running from one to one hundred. A discrete value that reflects the proportionate distance of the point selected from one end of the VAS is determined. For instance, a patient answer provided on a ten-centimeter-long VAS 40 would be rounded to the nearest millimeter and the distance 47 from the leftmost endpoint 42 would internally represent the patient's response. Other numeric scales or forms of quantifying a VAS response are possible. The discrete values representing each VAS answer are then evaluated against qualitative wellness criteria to determine patient risk, as further described below with reference to FIG. 5.

### Method

Self-assessment data obtained via a VAS can be combined with other data sources to evaluate patient well-being. FIG. 4 is a process flow diagram showing a method 60 for performing remote patient risk assessment through a VAS 40. The method is performed as a series of process steps by a communicator 15, or general purpose programmable computing device, such as a personal computer, cellular telephone, or other network-capable device.

Patient status through self-assessment is evaluated through a pair of recurring stages. During the first stage, patient data is measured and collected (operation 64) from a range of data sources 61-63. The data sources include implantable, extra-corporeal, and monitored sensor data 61 that record physiometry, environmental data, and parametric information; VAS data 62; and other quantitative and qualitative data sources 63, including conventional questionnaires and external resources, such as remote healthcare provider databases and third party references. The VAS data 62 is paired with the data from the other data sources 61, 63, which can corroborate any findings of risk against quantitative and qualitative wellness criteria. Still further sources of both objective and subjective patient data are possible.

During the second stage, the patient data is analyzed to determine patient risk (operation 65) and health alerts are created (operation 66), as further described below beginning with FIG. 5. Briefly, however, data analysis can include preprocessing the patient data to screen or eliminate cumulative or outlier values and deriving indirect physiometry, including formulating multivariate and trending values. In addition to risk assessment, the patient data, including VAS scores, can also be evaluated for the occurrence of one or more chronic or acute health conditions, such as described in related, commonly-owned U.S. Pat. No. 6,336,903, to Bardy, issued Jan. 8, 2002; U.S. Pat. No. 6,368,284, to Bardy, issued Apr. 9, 2002; U.S. Pat. No. 6,398,728, to Bardy, issued Jun. 4, 2002; U.S. Pat. No. 6,411,840, to Bardy, issued Jun. 25, 2002; and U.S. Pat. No. 6,440,066, to Bardy, issued Aug. 27, 2002, the disclosures of which are incorporated by reference. Finally, the data analysis can include post processing activities, which can include instructing the patient to unilaterally adjust his medications or by adjusting sensors or data thresholds. Other forms of patient analysis and processing are possible.

Raw patient responses to a VAS-provided question must first be objectified and evaluated before being considered with or against other patient data sources. FIG. 5 is a flow diagram showing a VAS processing routine 70 for use in the method of FIG. 4. The outputs from the routine are provided as VAS data 62.

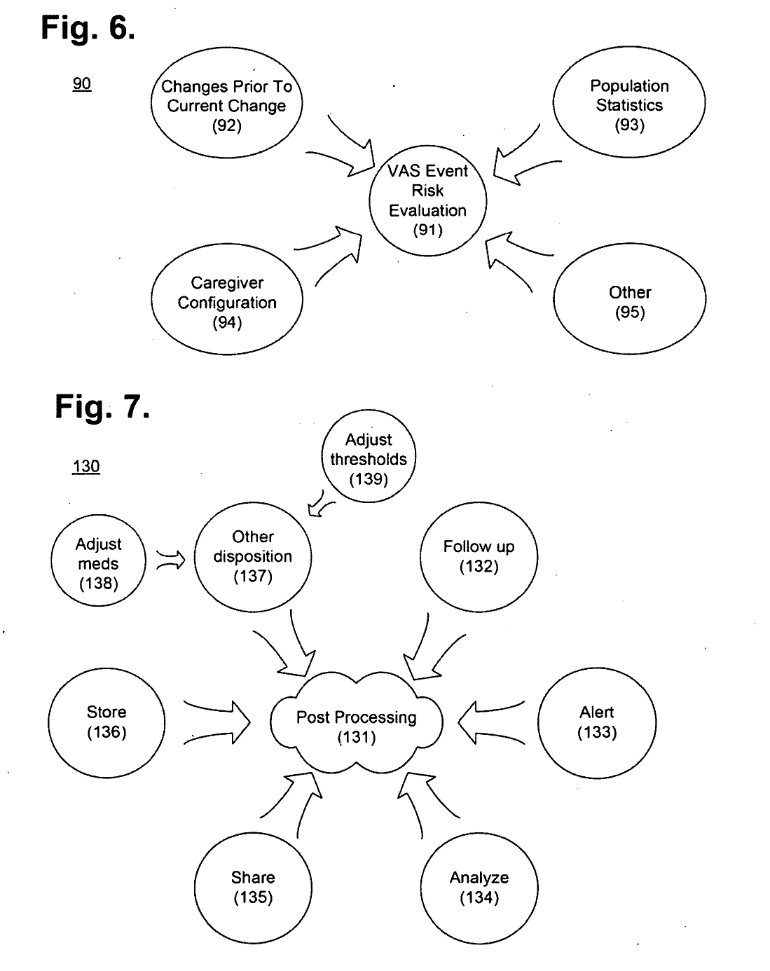

Questions or queries intended to solicit subjective, qualitative responses from a patient about a physiological condition or other area of caregiver interest are paired with a corresponding VAS. Each query or question 41 is separately processed through a sequence of steps. First, the query or question and corresponding VAS are displayed through the user interface 22 of a patient communicator 15 (shown in FIG. 1) (step 71). In response to the query or question, the patient 11 selects a point 46 on the VAS 40, which is accepted through the user interface 22 (step 72). The response is quantized by determining the distance of the point selected from the end of the VAS 40 and finding a fixed value in proportion to the distance (step 73). If appropriate, the change between current and previous VAS responses values is determined and stored (step 74). No changes would be found if, for instance, the query or question was being asked for the first time. Insignificant changes generally require no further processing (step 75). However, a significant change, such as a 20% difference over the most recent previous value may require event risk evaluation (step 76), as further described below with reference to FIG. 6. Processing continues in similar fashion for the remaining queries and questions. In a further embodiment, a patient medical device is interrogated contemporaneous to the answering of the query by the patient, and event risk is corroborated using device-recorded data. Other processing steps or thresholds are possible.

Generally, a significant change in a VAS-received response is only but one indicator of patient well-being. Considered in isolation, a VAS value can be evaluated against qualitative wellness criteria to identify a status quo or change in patient condition or can be trended against earlier observed responses. This type of basic evaluation may be helpful to assess patient risk, and factoring other patient data, including qualitative and quantitative, into risk evaluation can both corroborate and shed light on patient well being. FIG. 6 is a data flow diagram showing event risk evaluation 90 for use with the routine 70 of FIG. 5. Other factors, when considered in combination with a significant VAS change, may signal that the patient may be at risk for an event occurrence (operation 91). For instance, heart failure decompensation is frequently indicated by qualitative indications, such as respiratory distress, reduced exercise capacity, and cardiac palpitations, which present over time. Several VAS values as well as physiological data may need to be considered in combination to fully determine patient wellness.

Additionally, significant changes in VAS responses can be compared to other VAS changes (operation 92) that have occurred prior to the current change. An ongoing pattern of significant VAS changes can indicate a trend, which can provide credible indications that an underlying physiometric concern may be present. Other findings relating to current and prior VAS changes are possible.

In addition, VAS data is inherently subjective and personal to a particular patient 11. As a result, individual VAS measurements should only be compared to VAS data from other patients with caution. Notwithstanding, population statistics (operation 93) may be considered in respect to a significant VAS change for a specific patient, particularly where the VAS change is evaluated as a trend and not as a discrete data point viewed out-of-context. The VAS value can be weighted relative to the population statistics. A significant VAS change may then be found indicative of a potential event occurrence when observed for a similarly situated patient population. Other findings relating to population statistics are possible.

Moreover, a caregiver may configure or tailor (operation 94) the VASs provided to a particular patient 11. For example, a patient may require accommodations for impaired cognition, language, or reading difficulty. A caregiver might also desire more particularized answers than normally collected for other patients, such as on a more frequent or disorder-specific basis. Other findings relating to caregiver configuration are possible. Still other factors (operation 95) relating to event risk evaluation are possible.

### Post Processing

Results of a self-assessed VAS-based evaluation can be used to improve or modify patient care through post processing. FIG. 7 is a data flow diagram showing post processing of a remote patient self-assessment 130 for use with the method 60 of FIG. 4. Post processing affords trend identification and caregiver review.

Post processing (operation 131) can commence following analysis of individual VAS self-assessment responses or based collectively on a full VAS data set. Post processing can include follow up with the patient or custodians charged with day-to-day patient care (operation 132). Post processing can also include generating an alert (operation 133) to the physician or caregiver responsible for the patient. The alert can include indications of perceived risk of an event occurrence as identified through event risk evaluation, described above with reference to FIG. 6. Post processing can also include analyzing the patient's qualitative and quantitative data in detail (operation 134), such as by the server 18 (shown in FIG. 1) or other external system; sharing the analysis and event risk generation (operation 135) or storing the analysis (operation 136) in combination with other patient data. Still further post processing dispositions (operation 137) are possible. For instance, VAS values can be trended over time with identifiable trends displayed. Based on a significant trend lasting at least a predetermined number of days, the patient could be asked to directly self-adjust his medications (operation 138) based on a chart or guidance previously prepared by his caregiver, such as provided in Table 1. For example, if VAS values relating to heart failure decompensation increase, while intrathoracic total impedance (ITTI) values decrease, diuretics may be adjusted. Similarly, if the same VAS values increase along with resting heart rate, beta blocker medication may be adjusted. To guard against patients linking VAS results to medication adjustments, notifications to self-modify medication dosing are sent through the remote patient management system, which triggers under caregiver instructions or by heuristic analysis. One method for modifying medication based on patient-provided symptoms is described in Teresa M. Mueller et al., Telemanagement of Heart Failure: A Diuretic Treatment Algorithm For Advanced Practice Nurses, 31 Heart & Lung 340 (2002).

TABLE 1

| VAS Value Change | Diuretics | Beta Blocker |
|---|---|---|
| (over baseline) | (increase or decrease) | (increase or decrease) |
| 10% | — | 10 mg |
| 20% | — | 15 mg |
| 30% | 2x mg | 20 mg |

Further post processing dispositions (operation 137) can also include adjusting the thresholds used by sensors to analyze data (operation 139). For instance, VAS values could be feed into a heart failure decompensation analysis, which would enable, adjust, or disable thresholds based on evaluated patient risk. In particular, if the VAS values indicate that the patient has been feeling worse lately, heart failure decompensation-related sensors could be enabled or have their sensitivity increased. Conversely, if the VAS values indicate that the patient has been feeling better lately, the heart failure decompensation-related sensors could have their sensitivity decreased or be disabled. The remote patient management system would operate in a “smart” fashion to request or monitor data on an as-needed basis, thereby improving sensitivity and lowing false positive rates.

While the invention has been particularly shown and described as referenced to the embodiments thereof, those skilled in the art will understand that the foregoing and other changes in form and detail may be made therein without departing from the spirit and scope of the invention.

What is claimed is:

1. A system for performing remote patient risk assessment through a visual analog scale, comprising:
a visual analog scale comprising a gradient and descriptors for a continuous range of responses to a query;
a user interface operable by a remotely-managed patient, comprising:
a display configured to provide the query and the visual analog scale to the patient; and
an input control configured to accept an answer to the query comprising a point subjectively selected by the patient along the gradient;
a collection module configured to quantify the answer into a discrete value proportionate to a position of the point along the gradient determined from one of the ends; and
an analysis module configured to assess a risk to the patient comprising one of a status quo and change in condition by evaluating the discrete value against qualitative wellness criteria.

2. A system according to claim 1, wherein the risk is paired with other patient data that was obtained contemporaneous to the answer to the query, further comprising:
an evaluation module configured to corroborate the risk by evaluating the other patient data against one of quantitative wellness criteria and further qualitative wellness criteria.

3. A system according to claim 2, further comprising:
a device interface configured to periodically interrogate a patient medical device by uploading recorded data stored on the patient medical device, wherein at least part of the recorded data is designated as the other patient data.

4. A system according to claim 2, wherein the other patient data comprises at least one of physiometry, environmental data, and parametric information and further wherein the patient medical device is selected from the group comprising a pacemaker, implantable cardioverter defibrillator, biventricular pacemaker, implantable sensor, and implantable monitor.

5. A system according to claim 1, further comprising:
a post processing module, comprising modules configured to perform one or more of patient follow up, generating an alert, analyzing the risk, sharing the risk with others, storing the answer to the query, and providing notification to the patient to modify medication.

6. A system according to claim 5, wherein a dosage of the medication is modified by the patient based on a change of the discrete value, as compared with at least one of a baseline and previous discrete values.

7. A method for performing remote patient risk assessment through a visual analog scale, comprising:
defining a visual analog scale comprising a gradient and descriptors for a continuous range of responses to a query;
providing a user interface for a remotely-managed patient, comprising:
providing the query and the visual analog scale to the patient; and
accepting an answer to the query comprising a point subjectively selected by the patient along the gradient;
quantifying the answer into a discrete value proportionate to a position of the point along the gradient determined from one of the ends; and
assessing a risk to the patient comprising one of a status quo and change in condition by evaluating the discrete value against qualitative wellness criteria.

8. A method according to claim 7, further comprising:
pairing the risk with other patient data that was obtained contemporaneous to the answer to the query; and
corroborating the risk by evaluating the other patient data against one of quantitative wellness criteria and further qualitative wellness criteria.

9. A method according to claim 8, further comprising:
periodically interrogating a patient medical device by uploading recorded data stored on the patient medical device; and
designating at least part of the recorded data as the other patient data.

10. A method according to claim 8, wherein the other patient data comprises at least one of physiometry, environmental data, and parametric information and further wherein the patient medical device is selected from the group comprising a pacemaker, implantable cardioverter defibrillator, biventricular pacemaker, implantable sensor, and implantable monitor.

11. A method according to claim 7, further comprising:
post processing the risk comprising performing one or more of patient follow up, generating an alert, analyzing the risk, sharing the risk with others, storing the answer to the query, and providing notification to the patient to modify medication.

12. A method according to claim 11, wherein a dosage of the medication is modified by the patient based on a change of the discrete value, as compared with at least one of a baseline and previous discrete values.

13. A system for integrating qualitative assessment into remote patient management through a visual analog scale, comprising:
a query associated to an indication of at least one physiological condition;
a visual analog scale comprising a linear gradient and, at each end, descriptors for a range of subjective and continuous responses to a query;
an interrogation module configured to obtain assessment data for a remotely-managed patient, comprising:
a device interface configured to periodically interrogate a medical device of the patient and to receive stored data recorded by the medical device on a continuous basis; and
an interactive user interface for the patient, comprising:
a display configured to present the query with the visual analog scale; and
an input control configured to accept an answer to the query comprising a point selected by the patient between the ends of and along the linear gradient;
an array of sensors configured to determine a distance of the point from one end of the linear gradient;
a collection module configured to quantify the distance as a fixed value in proportion to the distance; and
an analysis module configured to assess a risk to the patient comprising one of a status quo and change in condition by analyzing the stored data and the fixed value against the at least one physiological condition to represent patient wellness.

14. A system according to claim 13, wherein at least one of population statistics and prior changes in condition to the patient are incorporated into the indication, further comprising:
an evaluation module configured to weight the fixed value relative to the indication as part of analysis of the risk to the patient.

15. A system according to claim 13, wherein the query is configured specifically for the patient, comprising one or more of accommodations for impaired cognition, language, or reading difficulty.

16. A system according to claim 13, further comprising:
a trend module configured to identify a trend in a plurality of the answers to a same query provided to the patient over time; and
an alert module configured to generate a notice to the patient to unilaterally adjust medication prescribed to treat the at least one physiological condition.

17. A system according to claim 13, further comprising:
a threshold module configured to adjust thresholds to at least one of a medical device, sensor, and data evaluation upon determining that the risk comprises a change in condition.

18. A system according to claim 13, wherein the at least one physiological condition comprises heart failure decompensation, further comprising:
an evaluation module to form the query and the visual analog scale to relate to one or more of respiratory distress, reduced exercise capacity, and cardiac palpitations.

19. A method for integrating qualitative assessment into remote patient management through a visual analog scale, comprising:
associating a query to an indication of at least one physiological condition;
forming a visual analog scale comprising a linear gradient and, at each end, descriptors for a range of subjective and continuous responses to a query;
obtaining assessment data for a remotely-managed patient, comprising:
periodically interrogating a medical device of the patient and receiving stored data recorded by the medical device on a continuous basis; and
providing an interactive user interface for the patient, comprising:
displaying the query with the visual analog scale; and
accepting an answer to the query comprising a point selected by the patient between the ends of and along the linear gradient;
determining a distance of the point from one end of the linear gradient;
quantifying the distance as a fixed value ill proportion to the distance; and
assessing a risk to the patient comprising one of a status quo and change in condition by analyzing the stored data and the fixed value against the at least one physiological condition to represent patient wellness.

20. A method according to claim 19, further comprising:
incorporating at least one of population statistics and prior changes in condition to the patient into the indication; and
weighting the fixed value relative to the indication as part of analysis of the risk to the patient.

21. A method according to claim 19, further comprising:
configuring the query specifically for the patient, comprising one or more of accommodations for impaired cognition, language, or reading difficulty.

22. A method according to claim 19, further comprising:
identifying a trend in a plurality of-the answers to a same query provided to the patient over time; and
generating a notice to the patient to unilaterally adjust medication prescribed to treat the at least one physiological condition.

23. A method according to claim 19, further comprising:
adjusting thresholds to at least one of a medical device, sensor, and data evaluation upon determining that the risk comprises a change in condition.

24. A method according to claim 19, wherein the at least one physiological condition comprises heart failure decompensation, further comprising:
forming the query and the visual analog scale to relate to one or more of respiratory distress, reduced exercise capacity, and cardiac palpitations.
